(19) United States
(12) Patent Application Publication
(10) Pub. No.: US 2025/0114557 A1
(43) Pub. Date: Apr. 10, 2025

(54) TEMPERATURE-RELATED DROWSINESS MITIGATION STRATEGY

(71) Applicant: GENTHERM INCORPORATED, Northville, MI (US)

(72) Inventors: Nicola Gerrett, Ann Arbor, MI (US); Matt Mallette, Paris (CA)

(21) Appl. No.: 18/707,976

(22) PCT Filed: Nov. 8, 2022
(86) PCT No.: PCT/US2022/049277

§ 371 Date: May 7, 2024

Related U.S. Application Data
(60) Provisional application No. 63/283,710, filed on Nov. 29, 2021.

(51) Int. Cl. A61M 21/00; B60H 1/00; B60N 2/56; B62D 1/06

(52) U.S. Cl. CPC A61M 21/00; B60H 1/00742; A61M 2021/0066; A61M 2021/0083; A61M 2205/3303; A61M 2205/362; B60N 2/56; B62D 1/065

(57) ABSTRACT

A vehicle occupant drowsiness mitigation system includes a microclimate that has multiple thermal effectors that are configured to thermally condition an occupant. The system includes an input that is configured to provide a signal that is indicative of a drowsiness condition of the occupant. A controller is in communication with the input and the multiple thermal effectors. The controller is configured to regulate the multiple thermal effectors in response to the signal to mitigate the drowsiness condition. The controller has different mitigation levels that are configured to provide different thermal conditioning to the occupant using the multiple thermal effectors.

### CROSS-REFERENCE TO RELATED APPLICATION

This application claims priority to U.S. Provisional Patent Application No. 63/283,710 filed on Nov. 29, 2021.

### BACKGROUND

This disclosure relates to microclimate thermal conditioning of a vehicle interior environment for individual occupants. More specifically, the disclosure relates to a vehicle microclimate system and a method for discouraging or enhancing sleepiness for a particular occupant.

Heating, ventilation and cooling (HVAC) systems are widely used in the automobile industry to control the temperature within the vehicle to increase occupant comfort. Increasingly, vehicles have incorporated additional, auxiliary thermal conditioning devices or thermal effectors, such as heated and cooled seats and heated steering wheels. These thermal effectors are intended to further personalize and enhance occupant comfort.

Drivers often become drowsy while driving. Vibrations, beeping and flashing lights have been used in an attempt to mitigate driver drowsiness, but with limited success. Cold air is often used to keep the driver alert. For example, if aware of their drowsiness, the driver may roll down the windows to allow cool air in or turn up the air conditioning. Non-driving occupants in the vehicle may become uncomfortable due to the additional noise or cold air, especially when the non-driving occupant desires sleep.

### SUMMARY

In one exemplary embodiment, a vehicle occupant drowsiness mitigation system includes a microclimate that has multiple thermal effectors that are configured to thermally condition an occupant. The system further includes an input that is configured to provide a signal that is indicative of a drowsiness condition of the occupant. The system further includes a controller that is in communication with the input and the multiple thermal effectors. The controller is configured to regulate the multiple thermal effectors in response to the signal to mitigate the drowsiness condition. The controller has different mitigation levels that are configured to provide different thermal conditioning to the occupant using the multiple thermal effectors.

In a further embodiment of any of the above, the different mitigation levels include at least first and second levels. The second level includes enhanced thermal conditioning compared to the first level. The controller is configured to initiate the enhanced thermal conditioning in response to the signal and subsequently reduce the thermal conditioning relative to the enhanced thermal conditioning.

In a further embodiment of any of the above, the input is commanded by the occupant.

In a further embodiment of any of the above, the controller is configured to select a predetermined level from the different mitigation levels based upon the signal. The controller is configured to change between the different mitigation levels based upon a time interval.

In a further embodiment of any of the above, the input is an occupant drowsiness detector that is configured to monitor the occupant. The controller is configured to determine the mitigation level based upon information from the occupant drowsiness detector and select from the different mitigation levels based upon the information.

In a further embodiment of any of the above, the controller is configured to change between the different mitigation levels based upon a change in the information from the occupant drowsiness detector.

In a further embodiment of any of the above, the multiple thermal effectors include at least two of a footwell vent, a steering wheel, a seat bottom, a seat back, and a neck thermal conditioner.

In a further embodiment of any of the above, the system includes an HVAC system with an opposing side footwell vent. The footwell vent is provided on a same side as the occupant, and the footwell vent is regulated differently than the opposing side footwell vent by the controller in response to the drowsiness condition.

In a further embodiment of any of the above, the different mitigation levels correspond to different zones of the occupant. The different zones include a hand/arm zone, a foot/leg zone, a cushion zone, a back zone, and a head/neck zone.

In a further embodiment of any of the above, the occupant is a driver, and includes another occupant, and includes a vehicle occupant sleep enhancement system that has another microclimate that has at least one other thermal effector that is configured to thermally condition the other occupant. Another input is configured to provide another signal indicative of a desired sleep condition for the other occupant. The controller is in communication with the other input and the at least one other thermal effector. The controller is configured to regulate the at least one other thermal effector in response to the other signal to initiate a sleep mode for the other occupant. The controller is configured to provide thermal conditioning to the other occupant's feet using the at least one other thermal effector.

In another exemplary embodiment, a method of mitigating drowsiness of a vehicle occupant, the method includes detecting a drowsiness condition of an occupant, determining a drowsiness level based upon the detection, and regulating multiple thermal effectors using different mitigation levels that provide different thermal conditioning to the occupant based upon the determined drowsiness level.

In a further embodiment of any of the above, the detecting step is performed using a manual input by the vehicle occupant.

In a further embodiment of any of the above, includes a step of selecting a predetermined level from the different mitigation levels based upon the determining step, and the regulating step changes between the different mitigation levels based upon a time interval.

In a further embodiment of any of the above, the different mitigation levels include at least first and second levels. The second level includes enhanced thermal conditioning compared to the first level. The regulating step includes initiating the enhanced thermal conditioning and subsequently reduce the thermal conditioning relative to the enhanced thermal conditioning.

In a further embodiment of any of the above, the different mitigation levels correspond to different zones of the occupant. The different zones include a hand/arm zone, a foot/leg zone, a cushion zone, a back zone, and a head/neck zone.

In a further embodiment of any of the above, the method includes detecting a sleep condition for another occupant, and regulating at least one other thermal effector to initiate a sleep mode for the other occupant.

In another exemplary embodiment, a vehicle occupant sleep enhancement system includes a microclimate that has at least one thermal effector that is configured to thermally condition an occupant. The system further includes an input that is configured to provide a signal indicative of a desired sleep condition for the occupant. The system further includes a controller that is in communication with the input and the at least one thermal effector. The controller is configured to regulate the at least one thermal effectors in response to the signal to initiate a sleep mode for the occupant. The controller is configured to provide thermal conditioning to the occupant's feet using the at least one thermal effector.

In a further embodiment of any of the above, the system includes an HVAC system with an opposing side footwell vent. The footwell vent is provided on a same side as the occupant, and the footwell vent is regulated differently than the opposing side footwell vent by the controller in response to the drowsiness condition.

In a further embodiment of any of the above, the input is commanded by the occupant.

### BRIEF DESCRIPTION OF THE DRAWINGS

The disclosure can be further understood by reference to the following detailed description when considered in connection with the accompanying drawings wherein:

FIG. 1 is a schematic view of a vehicle having a microclimate system.

FIG. 2 is a schematic view of a controller of the microclimate system and example inputs provided to the controller for the vehicle of FIG. 1.

FIG. 3 is a schematic view of the controller in communication with macroclimate devices and microclimate devices for the vehicle of FIG. 1.

FIG. 4 depicts a drowsiness mitigation system for an occupant.

FIG. 5 is a flowchart of method of operating the system shown in FIG. 4.

The embodiments, examples and alternatives of the preceding paragraphs, the claims, or the following description and drawings, including any of their various aspects or respective individual features, may be taken independently or in any combination. Features described in connection with one embodiment are applicable to all embodiments, unless such features are incompatible. Like reference numbers and designations in the various drawings indicate like elements.

### DETAILED DESCRIPTION

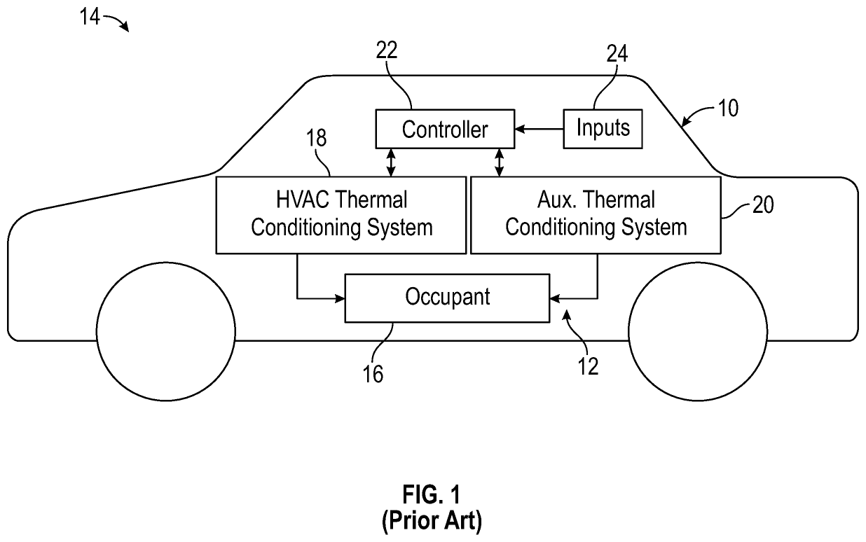

A vehicle 10, such as an automobile, is schematically shown in FIG. 1. The vehicle 10 includes a cabin or an interior space 12 for one or more occupants 16 that provides a vehicle interior environment in which the occupant experiences thermal comfort. The vehicle 10 is arranged in a vehicle exterior environment 14, which also can affect the thermal comfort of the interior space 12.

The disclosed system provides direct cooling (to mitigate drowsiness) or heating (to promote sleep) to specific body parts that are thermally sensitive and are linked to sleep onset. Depending upon the system and the desired outcome, one or more thermal effectors are suddenly or progressively changed to regulate the heat transfer at different body segments. This provides a system with the capability to change the microclimate conditions to address the occupant's level of drowsiness (typically a drive) or promote sleep of a non-driving occupant.

Scientific literature indicates that sleep onset latency is improved when distal body sites, especially the feet, are warm (Hardings et al. 2019). There is also evidence of people with impaired thermoregulatory abilities who experience strong levels of thermal discomfort have difficulties initiating sleep (Krauchi et al. 2008; 10.1111/j.1365-2869.2008.00678.x). There is also research indicating that whole body cold exposure can decrease sleep onset latency and increase wakefulness (Palca et al. 1986: 10.1152/jappl.1986.61.3.940). However, approaches for passenger vehicles are generally not effective and may adversely impact non-driving occupants within the vehicle.

Each occupant typically has a unique occupant personal comfort. That is, a particular occupant detects a level of thermal energy differently than another occupant. As a result, the exact same thermal environment within a vehicle may be perceived as comfortable by one occupant, but as uncomfortable by another occupant. To this end, this disclosure relates to regulating thermal effectors, such as climate-controlled seats (e.g., U.S. Pat. Nos. 5,524,439 and 6,857,697), head rest/neck conditioner (e.g., U.S. Provisional App. No. 62/039,125), climate-controlled headliner (e.g., U.S. Provisional App. No. 61/900,334), steering wheel (e.g., U.S. Pat. No. 6,727,467 and U.S. Pub. No. 2014/0090513), heated gear shifter (e.g., U.S. Pub. No. 2013/0061603, etc.) to achieve a personalized microclimate system. The referenced patents, publications and applications are incorporated herein by reference in their entirety. Portions of the vehicle's HVAC system may also be used to regulate the comfort (i.e., decrease or increase comfort) of a particular occupant.

In one example, the vehicle 10 includes a HVAC thermal conditioning system 18 and an auxiliary thermal conditioning system 20 (with microclimate devices, i.e., thermal effectors), which are in communication with a controller 22. Various inputs 24 may communicate with the controller 22 to affect and control operation of the HVAC thermal conditioning system 18 and/or the auxiliary thermal conditioning system 20. It should be understood that the vehicle may include more or fewer components than described below.

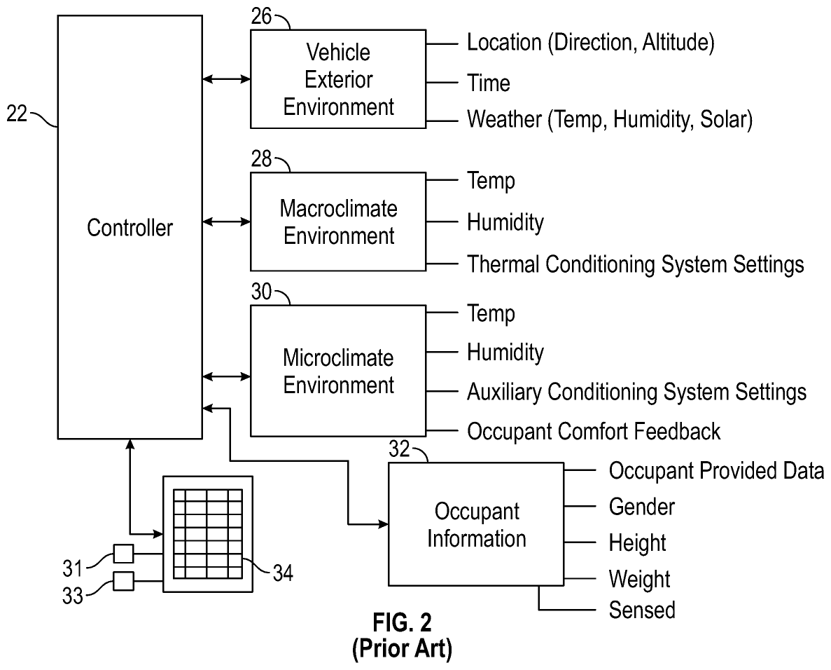

In one example microclimate system, the controller 22 receives various inputs via sensors and/or devices within the microclimate system, for example, from a vehicle exterior environment 26 shown in FIG. 2. The vehicle exterior environment 26 may include parameters such as vehicle location, vehicle direction and altitude, time of day and date, and weather-related parameters (outdoor temperature, outdoor humidity, and solar load on the vehicle).

A macroclimate environment 28 also communicates parameters to the controller 22. The macroclimate environment parameters may include interior temperature and/or humidity at one or more locations, and current HVAC system settings.

A microclimate environment 30 communicates parameters to the controller 22. The microclimate environment parameters may include temperature and/or humidity at one or more microclimate devices, auxiliary conditioning system settings, and occupant comfort feedback. Occupant comfort feedback may be provided when the occupant provides an input to control one of the microclimate devices, such as by changing the position of a switch.

Optionally, occupant information 32 is provided to the controller 22 for customizing and accounting for thermoreceptive differences between various occupants. It has been shown, for example, that women and men, generally speaking, react to heat and cold differently, with women reacting more severely and more quickly to cold and men reacting more quickly to heat. Additionally, the occupant information 32 can provide information for determining a thermal mass, heat capacity, and internal energy production rate. Occupant information 32 includes such information as sex, age, height, weight, and other occupant-provided data to provide a user profile. Then, for example, an initial default data set, or microclimate profile, could be defined during the customer vehicle purchase process, prior to any data being collected. Based on the default microclimate profile the system can begin the process of intuitively collecting data and then adjusting to individual's needs/wants based on the actual inputs by and use from the user over time. This initial microclimate profile could be based on any number of factors, including quantitative factors such as initial purchase location, driver characteristics (sex, height, weight, etc.), as well as qualitative factors, such as a survey where the respondent answers questions about their normal state of thermal comfort/stress. This information can be stored on a key fob or mobile device that is communicated to the controller 22. The user profile and learned microclimate profile can “move” with the occupant via the vehicle data link, the cloud, wireless transmission and/or smartphone, for example.

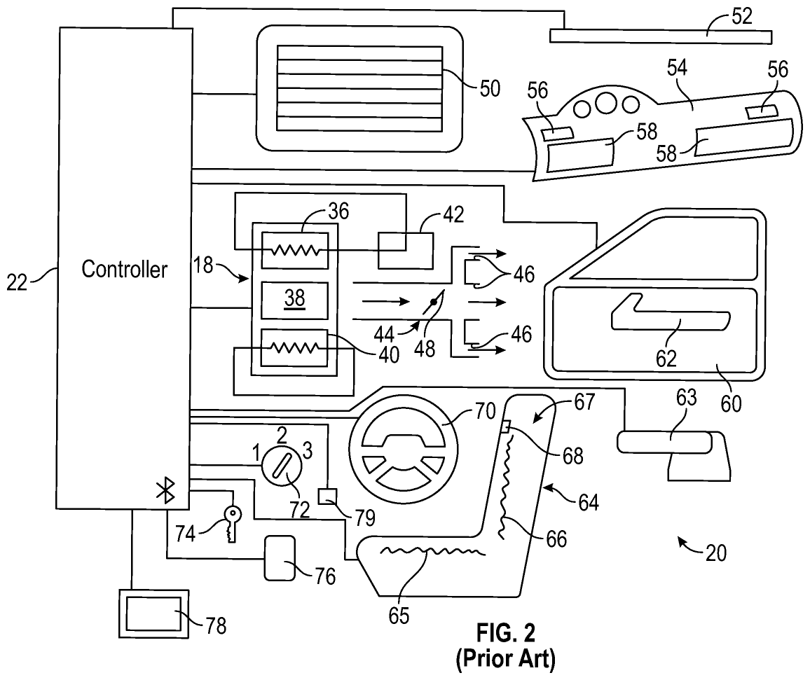

Sensed occupant information may also be provided (see, e.g., sensor 79 in FIG. 3), for example, by detecting occupant temperature. The sensor 79 may be used to detect drowsiness (e.g., heart rate, blinking, and/or head movement). In one example, the sensor 79 is a capacitive sensor in a steering wheel 70 (FIG. 3) used to detect heart rate via the driver's hands. In another example, the sensor 79 is a camera that is used to monitor eye and/or head movement. These sensed occupant personal comfort inputs are provided to the controller 22 for determining a perceived occupant personal comfort or drowsiness. The inputs can include one or more measured physiological parameters such as skin or other body temperatures such as a body core temperature. However, it should be understood that the disclosed system does not require a sensor for drowsiness detection in real time.

Multiple parameters from the vehicle exterior environment 26, the macroclimate environment 28, the microclimate environment 30, and the occupant information 32 may be stored in memory, such as one or more look-up tables 34. The memory may store information relating to one or more user profiles 31 and microclimate profiles 33 for various use scenarios corresponding to a particular user. The controller 22 may learn from adjustments to the microclimate system made by the occupant and update the microclimate profile 33 in the look-up tables 34 so that the occupant personal comfort may be anticipated and the microclimate system adjusted automatically. Interpolation of look-up table values or another suitable method can be used to determine settings between pre-existing set-points.

Referring in FIG. 3, an example HVAC thermal conditioning system 18 is in communication with the controller 22. The HVAC thermal conditioning system 18 includes a heat exchanger 36 in fluid communication with a heating loop connected to an engine 42. The engine 42 may include an internal combustion engine, an electric motor system, and/or a fuel cell. The engine 42 provides a heat source for the HVAC thermal conditioning system 18. An evaporator 40 is arranged in a cooling loop, which may include refrigerant and conventional air conditioning components typically found in a vehicle. It should be understood that a conventional HVAC system can instead be provided by one or more electrically operated microcompressors, if desired. A ventilation system 38, which provides fresh air to the HVAC system, may also be provided. The HVAC thermal conditioning system 18 typically includes ducting 44 providing multiple vents 46, including driver and non-driving occupant footwell vents. One or more valves 48 selectively control airflow from the HVAC system to the vents 46. These HVAC system components provide the macroclimate environment, but may be regulated in an individualized manner so as to provide a microclimate environment for a particular occupant.

The auxiliary thermal conditioning system 20 includes multiple microclimate devices, such as floor mat, a window defroster/defogger 50, a roof panel 52, one or more panels 58 in an instrument panel 54 (which may include vents 56), a door panel 60, a door arm rest 62, a center console armrest 63, a seat 64 having thermal elements 65, 66 and a neck conditioning device 67 having a vent 68, and/or a steering wheel 70. These microclimate devices are intended to regulate occupant thermal conditioning beyond what an HVAC system is capable by providing heating and/or cooling in close proximity to an occupant and thereby a more personalized microclimate environment within the surrounding interior environment. Heating and cooling can be provided by, for example, one or more heating elements, fans, thermoelectric devices, heat pumps, and/or microcompressors.

The inputs 24 are used to adjust the macroclimate environment and the microclimate environment through the controller 22 to achieve a desired occupant personal comfort. Inputs 24 include sensor signals and other inputs indicative of various parameters of the vehicle exterior environment 26, the macroclimate environment 28, and the microclimate environment 30. Inputs 24 further include one or more switches 72, a key fob 74 containing occupant information, a mobile device 76 containing occupant information and/or a display 78. The display 78 may visually display outputs or operating modes of the HVAC thermal conditioning system 18 and/or the auxiliary thermal conditioning system 20. The display 74 may also provide a means of input via a touchscreen, for example. The sensor 79 may provide real-time, sensed occupant information, such as drowsiness (e.g., heart rate, blinking, and/or head movement) temperature, moisture, humidity or other information. The display 74 may also include a “button” that can be operated by the driver to activate the disclosed drowsiness mitigation system.

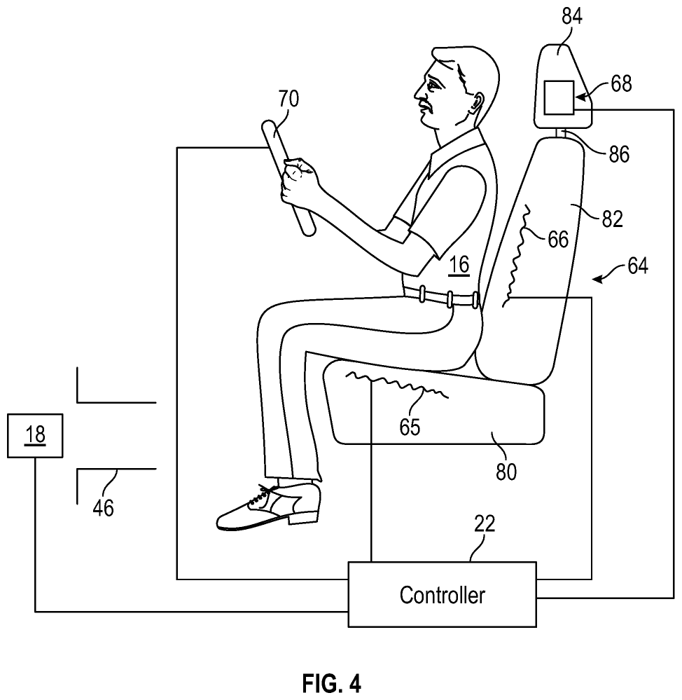

Generally, the vehicle microclimate system includes at least one microclimate device configured to be arranged within the interior space of the vehicle in close proximity to occupant zones such as a hand/arm zone, a foot/leg zone, an upper leg/buttocks zone, a back zone, and a head/neck zone. Referring to FIG. 4, the seat 64 may serve as one of the primary thermal effectors used to discourage or encourage sleep to an occupant. The footwell vent 46 as well as the steering wheel 70 may also be used to influence occupant thermal comfort. In the example shown, the seat includes a seat cushion 80 with a thermal effector 65 and a seat back 82 with another thermal effector 66, which may be separately controllable from the thermal effector 65. A head/neck conditioner 68 may be provided in a headrest 84 attached to the seat back 82 by posts 86 or integrated into the seat 64.

To mitigate drowsiness, the disclosed system can provide direct cooling to specific body parts that are thermally sensitive and are linked to sleep onset. Different thermal effectors (e.g., FIG. 4) may be regulated in response to a request by the occupant, or initiated automatically in response to detecting driver drowsiness with the sensor 79. The system can progressively change the heat transfer at different body segments by starting with fairly aggressive cooling to prevent sleep onset or by gradually increasing cooling until occupant drowsiness has been prevented. With either approach the system has the capability to progressively change the microclimate conditions according to the level of drowsiness detected.

A schematic overview of the process is shown below in the picture and an example of the different intervention options are shown in Table 1, below.

TABLE 1

| DROWSINESS | | |
|---|---|---|
| LEVEL | INTERVENTION | THERMAL EFFECTORS |
| Minor | Heat Withdrawal | Steering wheel-OFF |
| | | Footwell heat-OFF |
| | | Seat heating-OFF |
| Moderate-1 | Local Cooling at | All the above, + footwell cooling |
| Moderate-2 | Specific Body | All the above, + seat bottom |
| | Zones | cooling |
| Moderate-3 | | All the above, + seat back |
| | | cooling |
| Strong | Strong Localized | All of the above, + head/neck |
| | Cooling | cooling |

As can be appreciated from the Table above, the example different mitigation levels generally correspond to different zones of the occupant. Regarding heat withdrawal, warm environments can create feelings of drowsiness and can increase sleep onset, so removing any heat sources from the driver can be an effective first step. When melatonin, the hormone that regulates sleep is circulated around the body it causes vasodilation of blood vessels within the extremities, especially the feet. This causes an increase in foot skin temperature, dissipates heat to the environment causing core body temperature to drop, which helps trigger sleep onset. So, by locally cooling the feet, vasodilation can be prevented, keep foot skin temperature low and reduce the likelihood of sleep onset occurring. While the foot skin temperature is important for initiating sleep, the hands have a similar morphology and are also highly vascularized, meaning that by removing heat from the hands will also attenuate vasodilation and prevent the drop in core temperature that is required for sleep onset. Regarding the “moderate” indicators of drowsiness and its interventions, when individuals are uncomfortably cold, they have difficulties falling asleep. The moderate interventions provide an initial level of local mild discomfort and progressively increase the cooling to additional areas of the body and to areas that are progressively more thermally sensitive. The buttocks are a thermally sensitive body site to cooling local. By providing cooling to this region, an additional mild source of local thermal discomfort can be created. Cooling to an individual's head/neck (especially the face) generally creates the most discomfort. Since the neck is thermally very sensitive, increasing the airflow to the neck and down the spine, along with the backrest cooling on will deliver a strong thermal stimulus to create local thermal discomfort and keep the occupant alert.

In some circumstances a driver may feel the corresponding Intervention for a given Drowsiness Level is not strong enough. So, the driver may make some changes to the microclimate conditions to keep alert. In such instances, this information can be stored and used in the future to refine what the Intervention is for that driver at that Drowsiness Level.

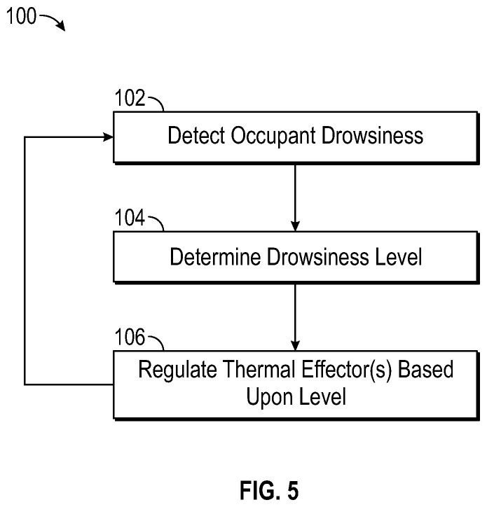

In operation, the system uses an example method (100 in FIG. 5) of detecting a drowsiness condition of an occupant (block 102). Drowsiness can be detected in response to a manual input of a drowsiness condition from the driver, for example, by the driver actuating a “drowsiness” button provided by a switch or touchscreen. Alternatively, the sensor 79 may detect driver drowsiness by monitoring driver behavior, which may include irregular steering inputs or lane departures.

In the case of automatic drowsiness monitoring and detection, a drowsiness level can be determined (block 104). Early signs of drowsiness may be yawning, whereas more intense feelings of drowsiness may be exhibited by prolonged eye closures and erratic driving. The controller 22 regulates multiple thermal effectors (e.g., footwell cooling, seat cooling, etc.) using different mitigation levels providing different thermal conditioning to the occupant based upon the determined drowsiness level (block 106). If a more proactive approach to drowsiness mitigation is desired, more effective mitigations strategies may be employed and then reduced from there (i.e., starting at “Strong” (i.e., enhanced) and moving toward “Minor”), however, this risks driver annoyance if the initial mitigation efforts are deemed too aggressive by the driver. Alternatively, the a less aggressive approach may be used initially (i.e., starting at “Minor” and moving toward “Strong”) and, if ineffective based upon driver monitoring, more aggressive mitigations measures can be used.

Where, for example, a real-time driver drowsiness monitoring sensor is not used, the system may default to a predetermined level from the different mitigation levels. Then, from the predetermined mitigation level, the system is regulated by changing between the different mitigation levels based upon a time interval. the different mitigation levels include at least first and second levels, the second level includes enhanced thermal conditioning compared to the first level, wherein the regulating step includes initiating the enhanced thermal conditioning and subsequently reduce the thermal conditioning relative to the enhanced thermal conditioning.

The above drowsiness mitigation is isolated to the driver so that other vehicle occupants are not affected. As an example, the footwell vents of the driver and non-driving occupant are operated independently. So, if footwell cooling is used to keep the driver awake, the non-driving occupant may still be supplied heated air, if desired.

This basic approach can also be used to promote sleep for a non-driving occupant. That is, the system may be used to create a microclimate solution around the occupant which will facilitate napping, typically by warming the occupant. Scientific literature indicates warm feet accelerates sleep onset, so the system may include a “nap mode” for non-driver occupants. Alternatively, if the driver decides that the warning of drowsiness is a concern and they want to prevent falling asleep at the wheel, they can be advised to pull over and initiate a “nap mode” that can be used for the driver when the vehicle is not operational. The driver could then pull over at a rest area for a brief nap, ensuring the driver wakes up refreshed when the journey is resumed.

To this end, the system detects a sleep condition for an occupant, for example, by the occupant requesting a “sleep mode” by selecting this option via a button or touchscreen, which provides a signal to the controller 22. The system then regulates at least one other thermal effector to initiate a sleep mode for the other occupant. In one example, the system increases the heating in the foot area (e.g., by operating a nearby HVAC vent 46). Other thermal effectors, such as seat heaters, may be used to increase occupant thermal comfort. Personalized settings also may be referenced to maximize comfort.

The controller 22 is in communication with the microclimate device. The controller is configured to determine an occupant personal comfort, for example, based upon the occupant information 30. The controller 22 commands the microclimate device in response to the occupant personal comfort to provide increased occupant comfort beyond what the HVAC system can provide, thereby fine-tuning the occupant's immediately surrounding environment.

It should be noted that a controller 22 can be used to implement the various functionality disclosed in this application. The controller 22 may include one or more discrete units. Moreover, a portion of the controller 22 may be provided in the vehicle 10, while another portion of the controller 22 may be located elsewhere. In terms of hardware architecture, such a computing device can include a processor, memory, and one or more input and/or output (I/O) device interface(s) that are communicatively coupled via a local interface. The local interface can include, for example but not limited to, one or more buses and/or other wired or wireless connections. The local interface may have additional elements, which are omitted for simplicity, such as controllers, buffers (caches), drivers, repeaters, and receivers to enable communications. Further, the local interface may include address, control, and/or data connections to enable appropriate communications among the aforementioned components.

The controller 22 may be a hardware device for executing software, particularly software stored in memory. The controller 22 can be a custom made or commercially available processor, a central processing unit (CPU), an auxiliary processor among several processors associated with the controller, a semiconductor-based microprocessor (in the form of a microchip or chip set) or generally any device for executing software instructions.

The memory can include any one or combination of volatile memory elements (e.g., random access memory (RAM, such as DRAM, SRAM, SDRAM, VRAM, etc.)) and/or nonvolatile memory elements (e.g., ROM, hard drive, tape, CD-ROM, etc.). Moreover, the memory may incorporate electronic, magnetic, optical, and/or other types of storage media. The memory can also have a distributed architecture, where various components are situated remotely from one another, but can be accessed by the processor.

The software in the memory may include one or more separate programs, each of which includes an ordered listing of executable instructions for implementing logical functions. A system component embodied as software may also be construed as a source program, executable program (object code), script, or any other entity comprising a set of instructions to be performed. When constructed as a source program, the program is translated via a compiler, assembler, interpreter, or the like, which may or may not be included within the memory.

The disclosed input and output devices that may be coupled to system I/O interface(s) may include input devices, for example but not limited to, a keyboard, mouse, scanner, microphone, camera, mobile device, proximity device, etc. Further, the output devices, for example but not limited to, a printer, display, macroclimate device, microclimate device, etc. Finally, the input and output devices may further include devices that communicate both as inputs and outputs, for instance but not limited to, a modulator/demodulator (modem; for accessing another device, system, or network), a radio frequency (RF) or other transceiver, a telephonic interface, a bridge, a router, etc.

When the controller 22 is in operation, the processor can be configured to execute software stored within the memory, to communicate data to and from the memory, and to generally control operations of the computing device pursuant to the software. Software in memory, in whole or in part, is read by the processor, perhaps buffered within the processor, and then executed.

It should also be understood that although a particular component arrangement is disclosed in the illustrated embodiment, other arrangements will benefit herefrom. Although particular step sequences are shown, described, and claimed, it should be understood that steps may be performed in any order, separated or combined unless otherwise indicated and will still benefit from the present invention.

Although the different examples have specific components shown in the illustrations, embodiments of this invention are not limited to those particular combinations. It is possible to use some of the components or features from one of the examples in combination with features or components from another one of the examples.

Although an example embodiment has been disclosed, a worker of ordinary skill in this art would recognize that certain modifications would come within the scope of the claims. For that reason, the following claims should be studied to determine their true scope and content.

What is claimed is:

1. A vehicle occupant drowsiness mitigation system, comprising:
a microclimate having multiple thermal effectors configured to thermally condition an occupant;
an input configured to provide a signal indicative of a drowsiness condition of the occupant; and
a controller in communication with the input and the multiple thermal effectors, the controller configured to regulate the multiple thermal effectors in response to the signal to mitigate the drowsiness condition, the controller having different mitigation levels that are configured to provide different thermal conditioning to the occupant using the multiple thermal effectors.

2. The system of claim 1, wherein the different mitigation levels include at least first and second levels, the second level includes enhanced thermal conditioning compared to the first level, the controller configured to initiate the enhanced thermal conditioning in response to the signal and subsequently reduce the thermal conditioning relative to the enhanced thermal conditioning.

3. The system of claim 1, wherein the input is commanded by the occupant.

4. The system of claim 3, wherein the controller is configured to select a predetermined level from the different mitigation levels based upon the signal, the controller is configured to change between the different mitigation levels based upon a time interval.

5. The system of claim 1, wherein the input is an occupant drowsiness detector configured to monitor the occupant, the controller configured to determine the mitigation level based upon information from the occupant drowsiness detector and select from the different mitigation levels based upon the information.

6. The system of claim 5, wherein the controller is configured to change between the different mitigation levels based upon a change in the information from the occupant drowsiness detector.

7. The system of claim 1, wherein the multiple thermal effectors include at least two of a footwell vent, a steering wheel, a seat bottom, a seat back, and a neck thermal conditioner.

8. The system of claim 7, comprising an HVAC system with an opposing side footwell vent, the footwell vent provided on a same side as the occupant, and the footwell vent regulated differently than the opposing side footwell vent by the controller in response to the drowsiness condition.

9. The system of claim 1, wherein the different mitigation levels correspond to different zones of the occupant, the different zones include a hand/arm zone, a foot/leg zone, a cushion zone, a back zone, and a head/neck zone.

10. The system of claim 1, wherein the occupant is a driver, and including another occupant, and comprising a vehicle occupant sleep enhancement system having another microclimate having at least one other thermal effector configured to thermally condition the other occupant; another input configured to provide another signal indicative of a desired sleep condition for the other occupant; and the controller in communication with the other input and the at least one other thermal effector, the controller configured to regulate the at least one other thermal effector in response to the other signal to initiate a sleep mode for the other occupant, the controller configured to provide thermal conditioning to the other occupant's feet using the at least one other thermal effector.

11. A method of mitigating drowsiness of a vehicle occupant, the method comprising:
detecting a drowsiness condition of an occupant;
determining a drowsiness level based upon the detection; and
regulating multiple thermal effectors using different mitigation levels providing different thermal conditioning to the occupant based upon the determined drowsiness level.

12. The method of claim 11, wherein the detecting step is performed using a manual input by the vehicle occupant.

13. The method of claim 12, comprising a step of selecting a predetermined level from the different mitigation levels based upon the determining step, and the regulating step changes between the different mitigation levels based upon a time interval.

14. The method of claim 13, wherein the different mitigation levels include at least first and second levels, the second level includes enhanced thermal conditioning compared to the first level, wherein the regulating step includes initiating the enhanced thermal conditioning and subsequently reduce the thermal conditioning relative to the enhanced thermal conditioning.

15. The method of claim 11, wherein the different mitigation levels correspond to different zones of the occupant, the different zones include a hand/arm zone, a foot/leg zone, a cushion zone, a back zone, and a head/neck zone.

16. The method of claim 11, comprising:
detecting a sleep condition for another occupant; and
regulating at least one other thermal effector to initiate a sleep mode for the other occupant.

17. A vehicle occupant sleep enhancement system, comprising:
a microclimate having at least one thermal effector configured to thermally condition an occupant;
an input configured to provide a signal indicative of a desired sleep condition for the occupant; and
a controller in communication with the input and the at least one thermal effector, the controller configured to regulate the at least one thermal effectors in response to the signal to initiate a sleep mode for the occupant, the controller configured to provide thermal conditioning to the occupant's feet using the at least one thermal effector.

18. The system of claim 27, comprising an HVAC system with an opposing side footwell vent, the footwell vent provided on a same side as the occupant, and the footwell vent regulated differently than the opposing side footwell vent by the controller in response to the drowsiness condition.

19. The system of claim 17, wherein the input is commanded by the occupant.
